(19) United States
(12) Patent Application Publication
(10) Pub. No.: US 2025/0135052 A1
(43) Pub. Date: May 1, 2025

(54) PROCESS FOR HEAT STERILIZATION OF TISSUE ADHESIVE LEADING TO IMPROVED SHELF LIFE AND PRODUCT UNIFORMITY

(71) Applicant: Aspire Biotech, Inc., Colorado Springs, CO (US)

(72) Inventors: Joshua Thomas Snow, Colorado Springs, CO (US); Ian Nigel Askill, Colorado Springs, CO (US); Lori Ann Campbell, Colorado Springs, CO (US)

(73) Assignee: Aspire Biotech, Inc., Colorado Springs, CO (US)

(21) Appl. No.: 18/836,435

(22) PCT Filed: Feb. 5, 2023
(86) PCT No.: PCT/US2023/012355

§ 371 Date: Aug. 7, 2024

Related U.S. Application Data
(60) Provisional application No. 63/372,012, filed on Feb. 7, 2022.

(51) Int. Cl. A61L 2/00; A61L 24/06

(52) U.S. Cl. CPC A61L 2/0023; A61L 24/06

(57) ABSTRACT

A process for sterilization of tissue adhesive is provided that includes heating a container containing a quantity of tissue adhesive to maximal temperature of between 180° C. and 230° C. for a Time Averaged Sterilization Temperature heat flow up to 500,000 C*min/mmol relative to the millimolar quantity of tissue adhesive. The container is cooled to sterilize the cyanoacry late-based tissue adhesive. A composition of matter results that includes a container with a hermetic seal. A quantity of sterilized tissue adhesive is provided within the container and has storage stability at standard temperature and pressure of at least 36 months.

### RELATED APPLICATIONS

This application claims priority benefit of U.S. Provisional Application Ser. No. 63/372,012 filed Feb. 7, 2022; the contents of which are hereby incorporated by reference.

### FIELD OF THE INVENTION

The present invention in general relates to tissue adhesives, and in particular to processes for sterilization of tissue adhesives leading to improved shelf life and product uniformity.

### BACKGROUND OF THE INVENTION

Medical grade glues, sealants, and adhesives (hereinafter referred to as tissue adhesives) are most commonly used to promote fast, cosmetically appealing, and cost-effective closure for surgical and traumatic wounds. They are also used to reinforce or protect skin, for treatment of varicose veins, or in lieu of surgical drapes, etc. Topical tissue adhesives seal wounds to prevent infection, prevent liquid loss, control bleeding, or approximate wound edges. In order to address various medical needs and develop the clinical properties needed to replace, repair, close and protect living tissues in a subject's body, the characteristics of tissue adhesives are carefully engineered and optimized for specific applications.

Tissue adhesives must be sterile and generally non-toxic and provide effective performance under a variety of changing or challenging conditions at the site of application such as moisture, bleeding, flexibility, and strength requirements. Tissue adhesives should also be easy to apply and fast drying. Tissue adhesives for skin closure, may be used in conjunction with meshes, sutures and staples, or independently to hold tissue together and serve as a barrier to the ingress of infectious agents or the loss of fluids.

The literature and regulatory filings describe several methods used to provide sterile tissue adhesives for clinical use. Gamma radiation was described by McDonnell in U.S. Pat. No. 5,530,037; heat by Kotzev in U.S. Pat. No. 5,874,044; electron beam radiation by Hickey in U.S. Pat. No. 6,143,805 and Greff in U.S. Pat. No. 6,248,800; and X-rays by Zhang in U.S. Pat. No. 8,652,510. The dose of sterilizing radiation or treatment required by the regulatory authorities to make the claim “sterile” is related to the amount of microbial contamination (often measured in colony forming units of CFU) in the device before sterilization. Hence, some manufacturers can go to significant lengths to reduce the contamination of their devices so that the sterilization treatment is less harsh and therefore less damaging to their product. In the extreme, some products are filtered with 0.2 micron filters intended to remove any microbial contamination and then packaged in pre-sterilized containers under aseptic conditions. One such product Histoacryl received FDA approval as a tissue adhesive under PMA P050013 on Feb. 16, 2007. However, this method of processing has significant risks and costs associated with it and is one of the very few medical devices that is not “terminally sterilized”.

A particular class of synthetic tissue adhesive is cyanoacrylate derived adhesives that are generally used for external tissue repair. Cyanoacrylate derived adhesives are fast acting, provide a strong bond, are typically waterproof, somewhat flexible, and do not require a secondary dressing. While some lower homologues of cyanoacrylate adhesives have a potential for toxicity and tissue inflammation, the longer-chain derivatives, such as butyl cyanoacrylate and 2-octyl cyanoacrylate, are less toxic, and have been approved by the Food and Drug Administration (FDA). Cyanoacrylate adhesive dries fast, effectively stops the bleeding, keeps out dirt and air, and typically stays in place until the wounded area is healed. However, while cyanoacrylate adhesives by themselves were originally not recommended for deep or jagged wounds or for use over areas of high mobility and flexion areas such as joints, they are now used in combination with meshes in those indications. In addition to its tissue adhesive indications, cyanoacrylate adhesives have also been FDA cleared as a barrier against common bacterial microbes. The longer chain cyanoacrylate products are often used topically to reinforce tissue closure and used for minor lacerations. Additionally, cyanoacrylate tissue adhesives have been approved for treatment of varicose veins and as a liquid embolic treatment system for cerebral ‘AVMs’ and are used in some countries for securing hernia meshes.

Another class of tissue adhesives cleared by the FDA for topical use, is the methylidene malonate series of reactive monomers.

The fast-acting properties of both of these classes of adhesives derive from extremely reactive monomer species which are triggered to polymerize by both free radicals and ions. Due to their high reactivity they have limited shelf lives, but these are further reduced by excessive exposure to moisture, heat, light or other forms of energy. This makes sterilization complicated because the majority of terminal sterilization methods use high energy, or moisture and heat, to kill microbes.

The challenge with sterilizing these reactive monomer tissue adhesives has been getting the monomer to survive the “energy shock” of sterilization and still have a commercially viable shelf life. Often, large amounts of potentially toxic stabilizers are added to prevent the monomer from polymerizing during sterilization, even so, the shelf lives become limited. It has been observed that non-sterilized samples of cyanoacrylate tissue adhesive may last for 20 years, but cyanoacrylate based adhesives that are sterilized have been observed to have a room temperature shelf life of 2 years or less. The polymerization inevitably presents itself in an increased viscosity and the sterilized cyanoacrylate based adhesive goes solid or becomes too viscous to be expressed prior to the intended use. As such, viscosity is the aging parameter that usually limits shelf life of tissue adhesives and is the parameter by which aging is most commonly monitored.

An added complication of the current sterilization methods is the difficulty of getting uniform dose to all units being sterilized when having to maintain some economy of scale in a batch process. This inevitably results in some units getting more energy than other units just to ensure that all of the units receive the minimum required dose. This affects the performance properties of the product immediately, and variability across the batch can readily be seen. In addition, the units that have received more energy will “age” more rapidly and this will lead to even greater differences as the product ages. This has led to customers being aware of performance variability between units of product and even product complaints and slower product adoption due to variable performance.

The most common commercial tissue adhesives are batch sterilized in two stages. First the adhesive component is batch sterilized by heat in either glass vials or metal tubes. These are then placed in or with applicators to facilitate clinical use, and this is again packaged in an overpack to allow sterile “drop” into a sterile field. These assemblies are then batch sterilized again by gas, usually ethylene oxide gas, although chlorine dioxide is an alternative. This secondary gas batch sterilization cycle is at lower temperature and does little or nothing to further stress the reactive monomer components of the tissue adhesive. The typical batch heat sterilization cycles used for the first stage are 160° C. for two hours, or 170° C. for one hour. Of course, each of these commercial processes has significant heat up and cool down times associated with large ovens and large numbers of product that have to be processed to make a commercially viable process, which will increase the heat load on the product. Additionally, individual units within the batch experience the heating and cooling differently depending on where they are placed in the large commercial oven, this leads to significant performance variability across a batch. It is widely recognized that performance variability seen post-sterilization is magnified as aging occurs which exacerbates the unit to unit differences.

Fortunately, whereas chemical processes typically follow Arrhenius in accelerating by a factor of about 2 for every 10° C. increase in temperature, this is not the case for the death of infectious microbial agents which die at a much faster rate as temperatures increase closer to 200° C. Hence, this invention utilizes the determination that sterilization is independent of accepted reaction kinetics at elevated temperatures, and the lower heat load of a higher temperature for a shorter sterilization time can be used to improve the performance of certain materials that need to be sterilized and are sensitive to degradation by heat.

Currently practiced heat sterilization methods for cyanoacrylate tissue adhesives in glass ampules largely assume “Arrhenius-like” reaction kinetics whereby every 10° C. increase in temperature doubles the reaction rate (or “kill rate” in this case). Hence, processes are typically 160° C. for 120 minutes, or 170° C. for 60 minutes to achieve the required “overkill” levels of sterilization. Without the heat-up and cool down time, this is equivalent to a Time Averaged Sterilization Temperature (TAST) heat flow of 2,231,947 C*min/mmol (assuming ambient=25° C.).

With the heat-up and cool down time the TAST heat flow is a massive 4,408,289 C*min/mmol. These high heat loads and the differential between them explain both the deleterious effects of sterilization and the variability across the batch.

Therefore, there is a need to develop a sterilization process that has a minimal effect on the viscosity of the tissue adhesive. There also exists a need for sterilized cyanoacrylate-based adhesives that have an extended shelf life and greater uniformity relative to existing sterilized cyanoacrylate-based adhesives.

### SUMMARY OF THE INVENTION

A process for sterilization of cyanoacrylate based tissue adhesive is provided that includes heating a container inclusive of a quantity of tissue adhesive to maximal temperature of between 180° C. and 230° C. for a Time Averaged Sterilization Temperature heat flow of less than 500,000 C*min/mmol (excluding heat-up and cool down time) relative to the quantity of tissue adhesive. The container is cooled after sterilizing the tissue adhesive.

A composition of matter results that includes a container with a hermetic seal. A quantity of sterilized tissue adhesive is provided within the container that has storage stability at standard temperature and pressure of at least 36 months.

### BRIEF DESCRIPTION OF THE DRAWINGS

The present invention is further detailed with respect to the following non-limiting drawings.

FIG. 1 is a comparison of a standard current batch sterilization process using 170° C. for one hour, compared with the high temperature short duration process of the invention shown as a plot of temperature versus time;

FIGS. 2A-2C are temperature as a function of time plots of inventive sterilization profile for cyanoacrylate-based adhesive shown as temperature versus time in accordance with embodiments of the invention;

FIG. 3A is a schematic of a first inventive system:

FIG. 3B is a schematic of a second inventive system inclusive of a conveyor:

FIG. 4A is a graph of viscosity versus time (aging) for a set of samples of 100% butyl cyanoacrylate tissue adhesive formulation divided into the following groups: a non-sterile control group, a standard heat profile batch sterilized group, and a sample group treated with an inventive sterilization heat profile in accordance with embodiments of the invention;

FIG. 4B is a graph of the percentage difference from the non-sterile control group as shown in FIG. 4A of the viscosity versus time (aging) for the standard heat profile batch sterilized group and the sample group treated with the inventive sterilization heat profile in accordance with embodiments of the invention;

FIG. 5A is a graph of viscosity versus time (aging) for a set of samples of a blend of 80% butyl/20% octyl cyanoacrylate tissue adhesive formulation divided into the following groups: a non-sterile control group, a standard heat profile batch sterilized group, and a sample group treated with an inventive sterilization heat profile in accordance with embodiments of the invention;

FIG. 5B is a graph of the percentage difference from the non-sterile control group as shown in FIG. 5A of the viscosity versus time (aging) for the standard heat profile batch sterilized group and the sample group treated with the inventive sterilization heat profile in accordance with embodiments of the invention;

FIG. 6A is a graph of viscosity versus time (aging) for a set of samples of a 100% octyl cyanoacrylate tissue adhesive formulation divided into the following groups: a non-sterile control group, a standard heat profile batch sterilized group, and a sample group treated with an inventive sterilization heat profile in accordance with embodiments of the invention;

FIG. 6B is a graph of the percentage difference from the non-sterile control group as shown in FIG. 6A of the viscosity versus time (aging) for the standard heat profile batch sterilized group and the sample group treated with the inventive sterilization heat profile in accordance with embodiments of the invention; and

FIG. 7 is a graph depicting the temperature and time bounds of the invention in certain embodiments.

### DETAILED DESCRIPTION OF THE INVENTION

The present invention has utility as an improved process for sterilization of cyanoacrylate based tissue adhesives that results in a shelf stable product that has a shelf life that is similar to non-sterilized cyanoacrylate based adhesives. Embodiments of the inventive sterilization process provide a high heat for a short duration giving a low overall Time Averaged Sterilization Temperature (TAST). In a specific inventive embodiment, a peak temperature of 185° C. for 1 minute is applied. Embodiments of the inventive heating profile have a hotter peak temperature than traditional sterilization temperature profiles, but for a shorter period for a reduced area of heating. The present invention, in providing and range of heat flow values of between 400 C*min/mmol and 500,000 C*min/mmol is able to sterilize a tissue adhesive without inducing premature cure reaction thereof. The lack of premature cure reaction is manifest as a storage stability at standard temperature and pressure of at least 36 months and in specific embodiments of between 36 and 72 months. This range of heat flows being idealized and assuming that preceding heat up and succeeding cool down events are instantaneous. For the purposes of these calculations, the molar quantity relates to a quantity of pure butyl cyanoacrylate and the container in which the butyl cyanoacrylate resides is assumed to have instantaneous and infinite thermal transmissivity.

In specific inventive embodiments, shelf lives are achieved for sterilized cyanoacrylate based tissue adhesive formulations that are at least double that for current sterilized cyanoacrylate based tissue adhesive products with reduced levels of potentially toxic stabilizers. Depending on the stabilizers selected, shelf lives of 60 months or even 72 months are achieved.

Numerical ranges cited herein are intended to recite not only the end values of such ranges, but the individual values encompassed within the range and varying in single units of the last significant figure. By way of example, a range of from 0.1 to 1.0 in arbitrary units according to the present invention also encompasses 0.2, 0.3, 0.4, 0.5, 0.6, 0.7, 0.8, and 0.9; each independently as lower and upper bounding values for the range.

The following description of various embodiments of the invention is not intended to limit the invention to these specific embodiments, but rather to enable any person skilled in the art to make and use this invention through exemplary aspects thereof.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also, as used herein, “and/or” refers to and encompasses any and all possible combinations of one or more of the associated listed items, as well as the lack of combinations when interpreted in the alternative (“or”).

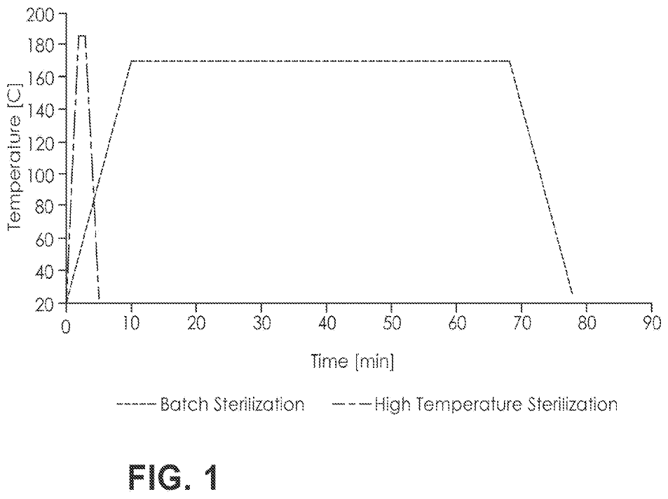

FIG. 1 is a comparison of a standard current batch sterilization process using 170° C. for one hour, compared with the high temperature short duration process of the invention shown as a plot of temperature versus time. The range on “heat flow” (factoring in heat up and cool down times) for a conventional batch process has a “heat flow” value of 4,408,289 C*min/mmol; absent preceding heat up and succeeding cool down the “heat flow” value is 2,231,947 C*min/mmol.

In contrast, an inventive process as shown in FIG. 1 with a hold temperature of 180° C., 240 s=157,549 C*min/mmol; while a hold temperature of 230° C., 0.5 s=419 C*min/mmol. According to a specific inventive process of 185° C., 1 min has a “heat flow” value of 381,670 C*min/mmol including heat up and cool down of the product. Without heat up and cool down the “heat flow” value is 40,481 C*min/mmol.

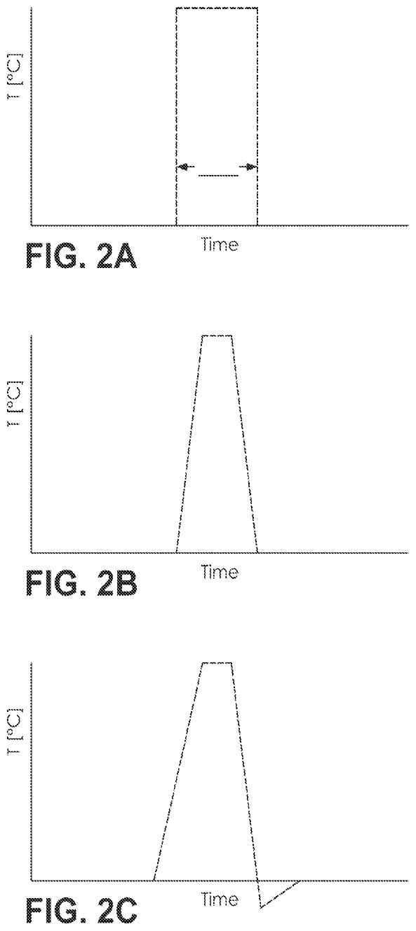

FIGS. 2A-2C are plots of an inventive sterilization profiles for cyanoacrylate based adhesive shown as temperature versus time with a peak temperature of 185° C. where the integrated area of theoretical profile Similar profiles exist for other tissue adhesives with routine experimentation applied to the specific formulation to achieve the results demonstrated hereafter for the exemplary material of cyanoacrylate-based tissue adhesives with the principal aspect of a short duration, burst of sterilization heating. Such other classes of tissue adhesives include acrylic bone cements and methylidene malonates. FIG. 2A is tightly controlled to achieve sterilization while achieving extended shelf life. This amounts to a controlled energy input, in units Time Averaged Sterilization Temperature heat input per mmol cyanoacrylate, as shown in FIG. 2A and not accounting for heat capacity of the container containing the cyanoacrylate. The value of which is readily determined experimentally and depends on factors such as the container material, wall thickness, cross-sectional shape.

The temperature profile zones may be achieved in separate heated baths for specific zones, or by moving between separate heated or cooled blocks or in separate zones of thermal chambers with rapidly circulating air to ensure rapid heat transfer.

The process was first verified using small vials as the containers in a cavity in a heating block by heating to 185° C. for 30 seconds, and then cooling. This led to sterilization of spore strips of Bacillus atrophaeus (population 106, D value 2).

To make this a commercially viable process that would be acceptable to the regulatory authorities, it needs to do more than reaching the temperature needed to kill all of the spores in a thermally resistant biological indicator. Regulatory authorities expect “overkill”—a level of sterilization that will ensure any organisms or spores thereof would have been killed many times over. In response, an inventive process in some embodiments expose containers of tissue adhesive to >185° C. for at least 1 minute.

A container operative herein illustratively includes those formed of borosilicate glass or quartz; metals such as stainless steel, aluminum or tin; ceramics such as alumina or titania; or high temperature tolerant plastics such as polyetherimide, polyether ether ketone, polytetrafluoroethylene, polybenzimidazole, polydicyclopentadiene, and copolymers in which any of the aforementioned, each alone or in combination constitute more than 50% of the copolymer.

For small glass containers containing less than 10 g of tissue adhesive, individual containers can be sterilized in a continuous process on a conveyor belt through a forced air oven with several zones and very close monitoring with thermocouples in the key zones to make sure the containers are experiencing the correct thermal profile for the correct amounts of time. A container containing a cyanoacrylate is heated rapidly to peak temperature of between 185 and 210° C. with heating and cooling ramps being so rapid as to approach the theoretical vertical ramps shown in FIG. 2A. The inventive process occurs in some embodiments absent the addition of chemical antimicrobials that can complicate cure and impact product shelf life. High ramp rates are achieved in some embodiments through techniques by plunging a container into a controlled temperature bath. Heat transfer within the container volume is further promoted by container rotation or agitation after, or in concert with a plunge event. It has been surprisingly found this type of shock process is as effective as conventional techniques while affording extended shelf life to the resulting cyanoacrylate, and the individual processing of units provides for less variability between units than a batch process.

If heat and time are apportioned correctly in the process according to the invention, no fundamental changes occur to the cyanoacrylate. The following processes are intended for the exemplary cyanoacrylate tissue adhesive sterilization:

Ultra-high temperature (UHT) sterilization 180-230° C. from 120 seconds at 180° C. to as little as 0.5 seconds at 230° C., followed by quench to 0° C. from 15 to 120 seconds to rapidly cool the container, as long as the container and contents are not damaged. A rate of cooling defined as 50% or greater than the rate of TAST heating is defined herein as shock cooling.

The range of TAST heat flow determined to be effective in sterilizing tissue adhesives with minimal damage by the inventive process, not including preceding heat-up and succeeding cool-down, is below 500,000 C*min/mmol. The calculated TAST heat flow for the example of 185° C. for 60 seconds has a TAST heat flow, excluding preceding heat-up and succeeding cool-down, of 40,481 C*min/mmol, as compared with a batch process of over 2 million C*min/mmol.

The information provided above shows clearly that temperature and time play very important roles and are product specific. Highly sensitive electronic devices for heat conduction and values for positive pressure/overpressure, which have been set in a highly sensitive manner, are used in the process and/or the device according to the present invention. Of particular significance according to the invention is the selection of suitable amounts of anionic inhibitors which can act in the gas phase at the sterilization temperatures in concert with the appropriate “dead volume” above the liquid in the ampoule by way of which pre-cure modification of the cyanoacrylate is prevented.

Sterilization of a container of cyanoacrylate is achieved with the aid of a multi-zone conveyor oven that allows for rapid heating of the container and maintaining it at the required sterilization temperature of ≥185° C. for 1 minute before cooling to a temperature to allow handling. The cooled containers are then further assembled into applicators as required depending on the intended use of the product and may be further over packaged and later sterilized by sterilizing gas or other terminal sterilizing process which will not damage the tissue adhesive.

With resort to a controlled batch or continuous process for a given container and amount of cyanoacrylate therein, a heat sensor, such as a thermocouple, a platinum resistance thermometer or a thermal luminescence spectroscopy system is used to monitor the thermal profile to which a container of cyanoacrylate is exposed. The sensor values indicative of cyanoacrylate temperature are transmitted to a digital display and in some embodiments converted to temperature units, thereby permitting all temperature values to be monitored and stored using a graphical display and a memory device in communication with the sensor. The proportional-integral-derivative (PID) values are amenable to a high degree of control as defined by ΔT is determined to be 0.1 or less.

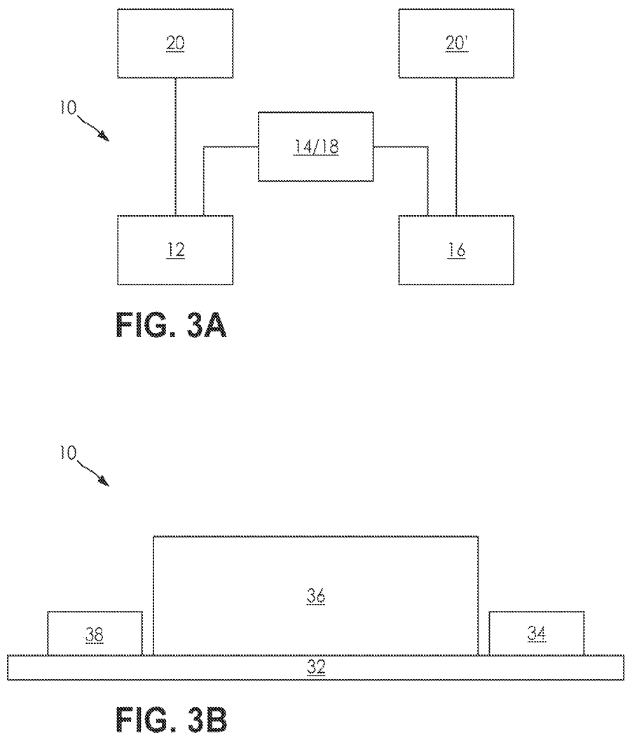

As shown in FIG. 3A, a system for performing the present invention is shown generally at 10, an ultra-high temperature (UHT) tank 12 is provided that is adapted to receive one or more containers of cyanoacrylate. It should be appreciated that uniformity in properties between containers regardless of whether processed in a batch or sequentially is of considerable importance. A robotic handling system 14 functions to transfer containers to a cooling tank 16. In some inventive embodiments, the tank 16 provides shock-cooling to the containers placed therein. In some inventive embodiments, an agitation system 18 promotes uniform thermal exposure within one, or both of the tanks 12,16. Temperature sensors 20 and 20′ monitor the temperature in tanks 12 and 16, respectively. In some inventive embodiments, the sensors 20 and 20′, each independently provides feedback control to modulate the temperature of the tanks 12 and 16, respectively.

As shown in FIG. 3B, a conveyor based system for performing the present invention is shown generally at 30, with containers moving on a conveyor 32 from right to left. Containers are loaded and enter a pre-heat warming zone 34 and thereafter enter a multi-zone oven 36 with temperature sensors therein. In some inventive embodiments, the temperature sensors are thermocouples. In still other inventive embodiments, two, three, or even more temperature sensors are provided per oven zone. In some inventive embodiments, the temperature sensors, such as thermocouples independently provide feedback control to modulate the temperature in a given zone of the multi-zone oven. After transiting from the multi-zone oven 36, the containers pass through a cooling zone 38 and thereafter are unloaded. It should be appreciated that single, like containers, with like lateral position on the conveyor 32 and a uniform motion to the conveyor readily imparts uniform thermal exposure to the containers.

The present invention provides a process of treating containers containing uncured cyanoacrylate with heat in a liquid or gaseous or vaporous medium in a plurality of steps. In an inventive process, the containers containing the cyanoacrylate are subjected to heating treatment, namely, ultra-high temperature processing by heating the containers containing the cyanoacrylate from 25° C. to between 180° C. and 230° C. and holding for as little as 0.5 seconds, to as much as 240 seconds, and then the containers containing the cyanoacrylate are cooled to 65° C. to 0° C.

The present invention also includes an apparatus for carrying out the process of the invention. The apparatus includes an ultra-high temperature unit which contains the medium and at least one section for heat transfer.

The invention is described in greater detail in the following with reference to exemplary embodiments shown in the drawings, without being limited thereby.

The present invention is further detailed with respect to the following examples that are not intended to limit the scope of the claimed invention, but rather to illustrate specific aspects of the invention.

### EXAMPLES

### Example 1

A comparative study is conducted to determine the effects of sterilization processes on the stability of cyanoacrylate tissue adhesives, as evidenced by changes in viscosity of a cyanoacrylate tissue adhesive over time. Three formulations of cyanoacrylate-based tissue adhesives are employed in the comparative study as summarized in Table 1 as follows:

TABLE 1
Study Formulations

| Study Legend | | Formulation |
|---|---|---|
| Butyl | Stability Study (Butyl) | 100% Butyl Cyanoacrylate 300 ppm |
| | | SO₂ 1000 ppm BHA* |
| Blend | Stability Study (Blend) | 80% Butyl/20% Octyl Cyanoacrylate |
| | | 75 ppm SO₂ 600 ppm BHA |
| Octyl | Stability Study (Octyl) | 100% Octyl Cyanoacrylate 80 ppm |
| | | SO₂ 500 ppm BHA |

*BHA is butylated hydroxyanisole

Three sets of three 0.8 ml samples are sealed in glass ampoules as the containers for each of the three formulations of Table 1:
A first set referred to as the control sample set from each of the three sets is set aside and is not subjected to a sterilization process.
A second set of three samples, referred to as the batch sample set, of the three formulations of Table 1 is sterilized using a traditional sterilization profile as illustrated in FIG. 1 with a peak temperature of 170° C. for 60 minutes. This has a TAST heat flow of 4,408,288 C*min/mmol with heat up and cool down for comparison.
A third set of three samples, referred to as the invention sample set, of the three formulations of Table 1 is sterilized using an embodiment of the inventive sterilization profile as illustrated in FIG. 2A with a peak temperature of 185° C. for 1 minute. For comparison, the TAST heat flow for this is ˜381,670 C*min/mmol.

Following sterilization of the two sample sets (batch sample set and inventive sample set), the three sample sets (control, batch, invention) are placed in a temperature chamber to undergo accelerated aging to simulate the equivalent of storage for two years to determine product stability of the three sample sets. The accelerated aging is conducted at 60° C., as determined through the use of the Arrhenius equation to calculate the real time (25° C.) equivalent aging (RTE) out to 24 months with a conservative Q10 factor of 2. The Q10 factor (“the rule of ten”) is used in accelerated studies, and is the factor by which the rate of reaction increases when the temperature is raised by 10° C. The Acceleration Rate (also called the Accelerated Aging Rate, Accelerated Aging Factor, or Acceleration Factor) is defined as the ratio of the real-world life-time to the test duration. The higher the acceleration rate, the less reliable the test is.

In the accelerated aging study of this experiment the effects of aging are measured by the changes in viscosity (measured in centipoise) of the adhesive formulations. As shown in the following tables and graphs for the three formulations of the adhesive (100% butyl cyanoacrylate, 80% butyl/20% octyl cyanoacrylate, or 100% octyl cyanoacrylate) there is the surprising result that with the inventive sterilization profile there is lack of increase in viscosity with aging relative to the batch sterilized formulation and is almost identical to the non-sterilized control.

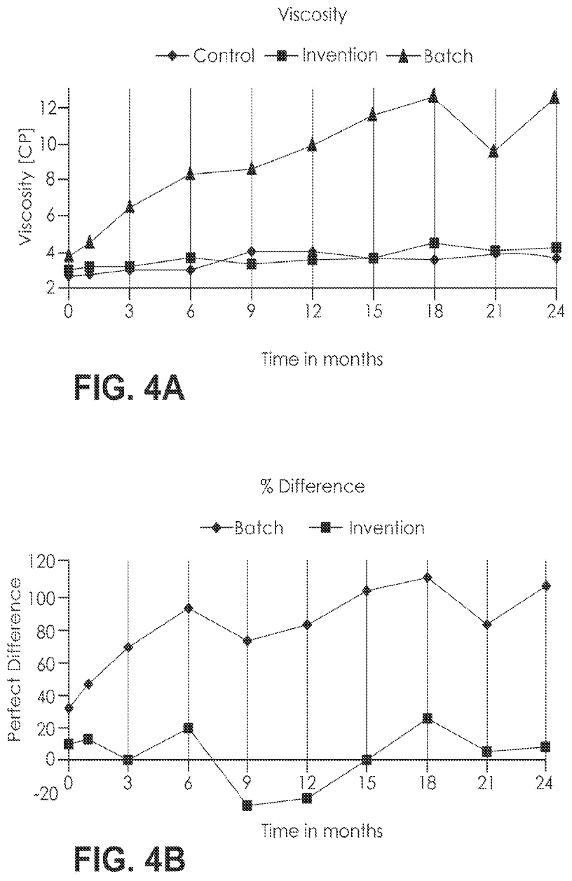

FIG. 4A is a graph of viscosity versus time (aging) for a set of samples of 100% butyl cyanoacrylate tissue adhesive formulation divided into the following groups: a non-sterile control group (Control), a standard heat profile batch sterilized group (Batch), and a sample group (Invention) treated with an inventive sterilization heat profile as outlined above. Table 2 summarizes the results of the sample trial for 100% butyl cyanoacrylate tissue adhesive formulation.

TABLE 2
Aging results for 100% butyl cyanoacrylate
tissue adhesive formulation

| | | | | % Diff | % Diff |
|---|---|---|---|---|---|
| RTE | Control | Invention | Batch | Batch | Invention |
| 0 | 2.7 | 3 | 3.74 | 32.3 | 10.5 |
| 1 | 2.76 | 3.14 | 4.44 | 46.7 | 12.9 |
| 3 | 3.09 | 3.15 | 6.46 | 70.6 | 1.9 |
| 6 | 2.98 | 3.62 | 8.33 | 94.6 | 19.4 |
| 9 | 3.93 | 3.28 | 8.57 | 74.2 | −18.0 |
| 12 | 4.03 | 3.48 | 9.93 | 84.5 | −14.6 |
| 15 | 3.61 | 3.57 | 11.57 | 104.9 | −1.1 |
| 18 | 3.52 | 4.52 | 12.62 | 112.8 | 24.9 |
| 21 | 3.83 | 4.00 | 9.56 | 85.6 | 4.3 |
| 24 | 3.78 | 4.05 | 12.56 | 107.5 | 6.9 |

As can be seen in the graph of FIG. 4A and Table 2, the viscosity of the batch that is sterilized with the standard heat profile displays a viscosity which steadily increases with time in months as expected and is indicative of premature aging of the 100% butyl cyanoacrylate tissue adhesive formulation. However, the sample group treated with the inventive sterilization profile that has a higher peak heat of 185° C. but for a shorter duration than the standard temperature profile had an aging profile that approximated the control group. FIG. 4B is a graph of the percentage difference from the non-sterile control group as shown in FIG. 4A of the viscosity versus time (aging) for the standard heat profile batch sterilized group and the sample group treated with the inventive sterilization heat profile. As can readily be seen in FIG. 4B, the viscosity of the sample treated with the inventive sterilization profile stays within a few percent of the non-sterilized control. Whereas the standard heat profile batch sterilized group has an increase in viscosity as the sample ages, and will eventually harden prior to its use.

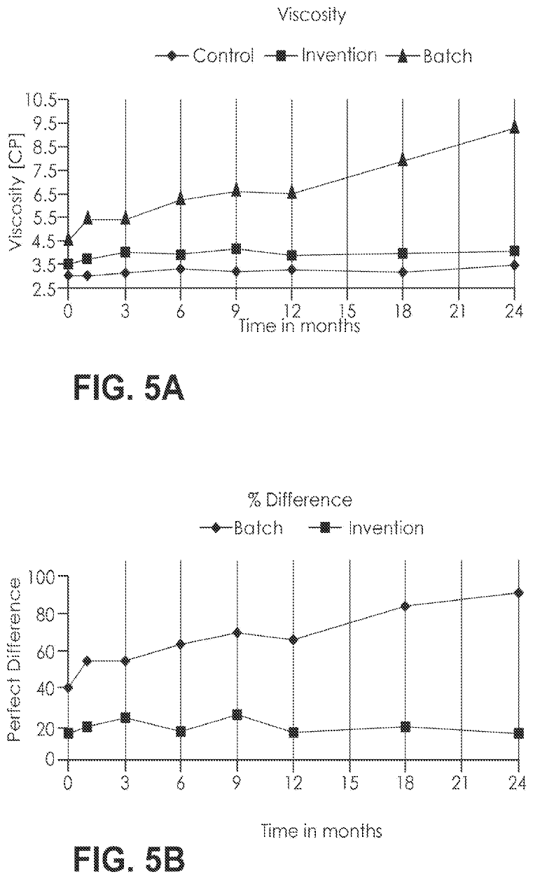

FIG. 5A is a graph of viscosity versus time (aging) for a set of samples of a blend of 80% butyl/20% octyl cyanoacrylate tissue adhesive formulation divided into the following groups: a non-sterile control group, a standard heat profile batch sterilized group, and a sample group treated with the inventive sterilization heat profile. Table 3 summarizes the results of the sample trial for the blend.

TABLE 3
Aging results for blend of 80% butyl/ 20% octyl
cyanoacrylate tissue adhesive formulation

| | | | | % Diff | % Diff |
|---|---|---|---|---|---|
| Months | Control | Invention | Batch | Batch | Invention |
| 0 | 3.06 | 3.58 | 4.68 | 41.9 | 15.7 |
| 1 | 3.13 | 3.79 | 5.47 | 54.4 | 19.1 |
| 3 | 3.16 | 4.02 | 5.51 | 54.2 | 24.0 |
| 6 | 3.31 | 3.91 | 6.36 | 63.1 | 16.6 |
| 9 | 3.24 | 4.19 | 6.68 | 69.4 | 25.6 |
| 12 | 3.34 | 3.91 | 6.63 | 66.0 | 15.7 |
| 18 | 3.31 | 4 | 8.06 | 83.6 | 18.9 |
| 24 | 3.52 | 4.10 | 9.38 | 90.9 | 15.1 |

As can be seen in the graph of FIG. 5A and Table 3, the viscosity of the batch that is sterilized with the standard heat profile displays a viscosity which steadily increases with time in months as expected and is indicative of premature aging of the blended cyanoacrylate tissue adhesive formulation. However, the sample group treated with the inventive sterilization profile that has a higher peak heat of 185° C. but for a shorter duration than the standard temperature profile had an aging profile that approximated the control group. FIG. 5B is a graph of the percentage difference from the non-sterile control group as shown in FIG. 5A of the viscosity versus time (aging) for the standard heat profile batch sterilized group and the sample group treated with the inventive sterilization heat profile. As can readily be seen in FIG. 5B, the viscosity of the sample treated with the inventive sterilization profile stays within a few percent of the non-sterilized control. Whereas the standard heat profile batch sterilized group has an increase in viscosity as the sample ages and will eventually harden prior to its use.

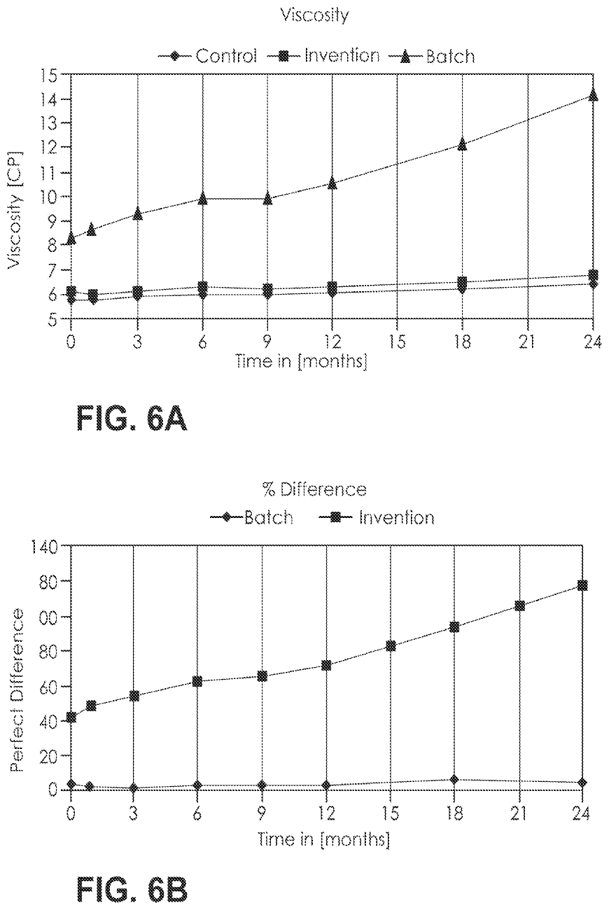

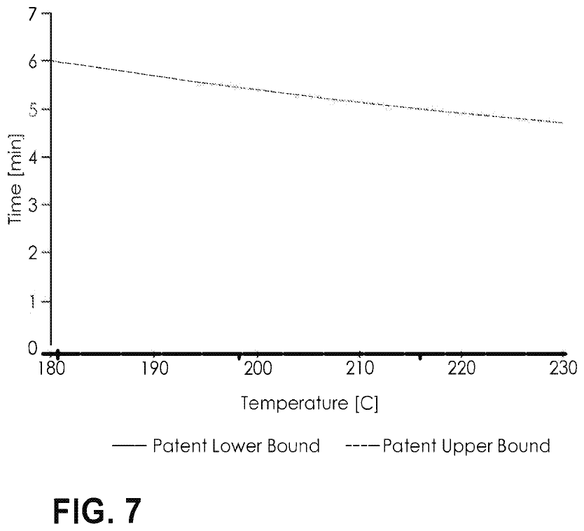

FIG. 6A is a graph of viscosity versus time (aging) for a set of samples of a 100% octyl cyanoacrylate tissue adhesive formulation divided into the following groups: a non-sterile control group, a standard heat profile batch sterilized group, and a sample group treated with an inventive sterilization heat profile. Table 4 summarizes the results of the sample trial for the 100% octyl cyanoacrylate tissue adhesive formulation.

TABLE 4
Aging results for blend of 100% octyl cyanoacrylate
tissue adhesive formulation

| | | | | % Diff | % Diff |
|---|---|---|---|---|---|
| RTE | Control | Invention | Batch | Batch | Invention |
| 0 | 5.82 | 6.05 | 8.34 | 42.5 | 3.9 |
| 1 | 5.81 | 5.93 | 8.7 | 49.2 | 2.0 |
| 3 | 5.99 | 6.06 | 9.31 | 55.1 | 1.2 |
| 6 | 6.02 | 6.22 | 9.91 | 63.6 | 3.3 |
| 9 | 5.94 | 6.16 | 9.93 | 66.0 | 3.6 |
| 12 | 6.08 | 6.25 | 10.52 | 72.0 | 2.8 |
| 18 | 6.16 | 6.48 | 12.1 | 94.0 | 5.1 |
| 24 | 6.4 | 6.75 | 14.14 | 117.7 | 5.3 |

As can be seen in the graph of FIG. 6A, and Table 4, the viscosity of the batch that is sterilized with the standard heat profile displays a viscosity which steadily increases with time in months as expected and is indicative of premature aging of the octyl cyanoacrylate tissue adhesive formulation. However, the sample group treated with the inventive sterilization profile that has a higher peak heat of 185° C. but for a shorter duration than the standard temperature profile had an aging profile that approximated the control group. FIG. 6B is a graph of the percentage difference from the non-sterile control group as shown in FIG. 6A of the viscosity versus time (aging) for the standard heat profile batch sterilized group and the sample group treated with the inventive sterilization heat profile. As can readily be seen in FIG. 6B, the viscosity of the sample treated with the inventive sterilization profile stays within a few percent of the non-sterilized control. Whereas the standard heat profile batch sterilized group has an increase in viscosity as the sample ages and will eventually harden prior to its use.

The experiment with three different formulations of cyanoacrylate based tissue adhesive conclusively showed that the inventive sterilization profile had little to no effect on the shelf life of the adhesive product.

A further example involves taking an ethoxyethyl cyanoacrylate tissue sealant thickened with 8% polymethyl methacrylate (PMMA) to a viscosity of approximately 120 cPs. The tissue sealant formulation is not designed for aggressive sterilization with high energy and so does not contain high levels of toxic stabilizers. 0.7 ml samples of this are sealed in onion skin glass ampules as the containers and exposed to simulated sterilization conditions by either batch sterilization or the conditions of the invention. The samples are cooled and stored refrigerated until their viscosities are tested. The simulated batch sterilization involves heating to 170° C. followed by cooling to ambient by turning off the oven and opening the oven door. The inventive sterilization condition involves rapid heating to 185° C., holding for one minute followed by cooling with fans.

The following table shows the viscosities (in centipoise) of a tissue sealant when exposed to the two different sterilization processes:

TABLE 5
Ethoxyethyl cyanoacrylate w/ 8% PMMA

| Sample # | Invention | Batch |
|---|---|---|
| 1 | 141.8 | 706.1 |
| 2 | 143.7 | *** |
| 3 | 143.5 | *** |
| 4 | 141.6 | *** |
| 5 | 142.0 | *** |
| 6 | 147.2 | *** |
| 7 | 143.9 | 552.8 |
| 8 | 144.5 | 649.9 |
| 9 | 143.7 | *** |
| 10 | 145.5 | 471.1 |
| Avg | 143.7 | 595.0 |
| Std Dev | 1.7 | 104.1 |

***Sample did not stabilize indicating the viscosity is > 1022 cP or particulate was present preventing measurement stabilization

As can readily be seen, sterilization by the inventive process increases the viscosity slightly, but the samples are still useable and the viscosities are very uniform. In contrast, the traditional sterilization method, yields product that was both too thick to spread, and/or very variable across the small batch. This is in line with user concerns that current product can be very variable in performance.

The four formulations used in the examples are just very simple examples of many possible cyanoacrylate or other adhesive formulations that could be sterilized by the process of this invention. The cyanoacrylates that the inventors have used in formulations have included homologues with side chains ranging from 2 carbons to 12 carbons, with and without branching and oxygen groups. The inventors and others skilled in the art are very familiar with the stabilizers, thickeners, plasticizers, colorants, accelerators and initiators that may be used to adjust the clinical performance of tissue adhesives. Many of these are listed at length in Zhang, however, the most commonly used free radical stabilizer is butylated hydroxyl anisole, and the most common anionic stabilizer is sulfur dioxide.

Other tissue adhesives have included methylidene malonate and its derivatives which are similarly susceptible to deterioration under sterilization conditions.

### CITED REFERENCES

Hawkins, Surgical Adhesive Compositions. U.S. Pat. No. 3,591,676
McDonnell, Sterilized Cyanoacrylate Adhesive Composition and a Method of Making such a composition. U.S. Pat. No. 5,530,037
Askill, Methods of Draping Surgical Incision Sites. U.S. Pat. No. 5,730,994 Kotzev, Heat Sterilization of Cyanoacrylate. U.S. Pat. No. 5,874,044
Hickey, Electron Beam Sterilization of Liquid Adhesive Compositions. U.S. Pat. No. 6,143,805
Greff, methods for Sterilizing Cyanoacrylate Compositions. U.S. Pat. No. 6,248,800
Zhang, Sterilized Liquid Compositions of Cyanoacrylate Monomer Mixtures. U.S. Pat. No. 8,652,510

Patent documents and publications mentioned in the specification are indicative of the levels of those skilled in the art to which the invention pertains. These documents and publications are incorporated herein by reference to the same extent as if each individual document or publication is specifically and individually incorporated herein by reference.

The foregoing description is illustrative of particular embodiments of the invention but is not meant to be a limitation upon the practice thereof. The following claims, including all equivalents thereof are intended to define the scope of the invention.

1. A process for sterilization of a tissue adhesive, said process comprising:
heating a container containing a quantity of said tissue adhesive to maximal temperature of between 180° C. and 230° C. for a Time Averaged Sterilization Temperature heat flow up to 500,000 C*min/mmol relative to said millimolar quantity of tissue adhesive; and
cooling said container to sterilize said quantity of said tissue adhesive.

2. The process of claim 1 wherein said cooling is shock cooling.

3. The process of claim 1 wherein said Time Averaged Sterilization Temperature heat flow should not exceed 250,000 C*min/mmol relative to said quantity of tissue adhesive.

4. A process of claim 1 wherein the tissue adhesive is one of an acrylic bone cement, a methylidene malonate, or a cyanoacrylate ester.

5. A composition of matter comprising:
a container with a hermetic seal;
a quantity of sterilized tissue adhesive within said container, said quantity of sterilized tissue adhesive has storage stability at standard temperature and pressure of at least 36 months.

6. The composition of matter of claim 5 wherein the storage stability is between 36 and 72 months.

7. The composition of matter of claim 5 wherein said container is formed of borosilicate glass, metal, ceramic, or a high temperature tolerant plastic.

8. The composition of claim 5 wherein said tissue adhesive is a cyanoacrylate.

9. The composition of claim 8 wherein said cyanoacrylate is butyl cyanoacrylate, octyl cyanoacrylate, or a combination thereof.

10. The composition of matter of claim 5 further comprising a free-radical stabilizer.

11. The composition of matter of claim 10 wherein said free-radical stabilizer is BHA, BHT, 4-methoxyphenol, hydroquinone, 4-ethoxyphenol, 3-methoxyphenol, catechol, or combinations thereof.

12. The composition of matter of claim 5 further comprising a preservative.

13. The composition of matter of claim 12 wherein said preservative is SO₂, boron trifluoride, or at least one strong acid of perchloric, sulfuric, hydrochloric, methane sulfonic, toluene sulfonic, phosphoric, or a combination thereof.

14. The composition of matter of claim 5 further comprising a thickening agent.

15. The composition of matter of claim 14 wherein said thickening agent is PMMA, polyvinyl acetate, poly cyanoacrylate polymers and copolymers, or a combination thereof.

16. The composition of matter of claim 5 further comprising at least one of a plasticizer, a citrate, a sebacate, a colorant, an accelerant, or a combination thereof.

17. The composition of matter of claim 5 formed by the process of claim 1.
